(19) United States
(12) Patent Application Publication
(10) Pub. No.: US 2010/0204462 A1
(43) Pub. Date: Aug. 12, 2010

(54) PIPETTE TIP WITH SEPARATION MATERIAL

(76) Inventors: Thomas Walter, Penzberg (DE); Herbert Harttig, Neustadt (DE); Maximilian Ruhstorfer, Luzern (CH); Martin Victor, Wolfratshausen (DE)

Correspondence Address: ROCHE DIAGNOSTICS OPERATIONS INC., 9115 Hague Road, Indianapolis, IN, 46250-0457, US

(21) Appl. No.: 12/571,581

(22) Filed: Oct. 1, 2009

(30) Foreign Application Priority Data
Oct. 13, 2008 (EP) 08017904.7

(51) Int. Cl. C07H 21/00; B01L 3/02; G01N 1/28

(52) U.S. Cl. 536/25.41; 422/100

(57) ABSTRACT

The present invention provides an attachment or extension for pipettes and pipette tips. The attachment comprises separation materials which are preferably suitable for isolating nucleic acids from liquids. The separation materials are present in the form of a filter disc in a holder which only slightly increases the pipette volume and thus generates little dead volume.

### RELATED APPLICATIONS

This application claims priority to European patent application EP 08017904.7 filed Oct. 13, 2008.

### FIELD OF THE INVENTION

The present invention provides an attachment or extension for pipettes and pipette tips. The attachment comprises separation materials which are preferably suitable for isolating nucleic acids from liquids. The separation materials are present in the form of a filter disc in a holder which only slightly increases the pipette volume and thus generates little dead volume.

BACKGROUND

Nucleic acids are currently purified by means of silica fleeces, glass particles or ion exchanger matrices.

With regard to fleeces they can be isolated manually or automatically in individual centrifugable columns or filter plates (96 or 384 wells). It is also possible to magnetically separate magnetic glass particles either manually or in various automated embodiments for example with 6, 8, 32, 48 or 96 samples per batch. In this case the magnetic separation takes place in a reaction vessel or a pipette tip.

In the respective processes additional steps are necessary to purely transfer liquids. They comprise steps such as centrifugation, filtration, magnetic separation or extraction. This results in a considerable expenditure of time and apparatus. Particularly the automation in a high throughput format is made very much more complicated, expensive, more likely to require maintenance and longer with regard to the time course due to the respective instrument modules.

Pipette tips are already known which can be used to purify analytes. Examples of this are the products of the Harvard Bioscience Company marketed under the name PrepTip which are also described in U.S. Pat. No. 6,416,716. In this case the wall of the pipette tip was coated at the lower outlet with certain affinity materials. In another embodiment chemically modified plastic material is used to manufacture (injection moulding) the pipette tip such that the wall of the pipette tip has affinity groups for binding analytes. A common feature of both embodiments is that the area for binding analytes is relatively small compared to porous membranes that are used nowadays. Also the diffusion paths when the analysis solution sweeps past are relatively large and hence the yield of purified analyte is too low and inadequate for high demands on yield.

Other pipette tips are filled with the solid phase at the lower outlet. They are used for example for the gel chromatography of DNA sequencing reactions to separate excess fluorescently labelled nucleotides which can interfere with a subsequent analysis. An example is described in U.S. Pat. No. 6,048,457. The National Scientific Supply Company offers so-called BioPack MicroColumn QuickKits. The tip of these pipette tips is filled with chromatographic material. Although the surface available for binding the analyte is enlarged in this case, carry-over of impurities from the samples and from the solutions and reagents that are used occurs into the eluate which contains the purified analyte due to the likewise large capillary volume of the chromatographic material.

The document DE 10 2005 053 463 describes a device and method for the automated isolation and purification of nucleic acids. The document EP 1 882 524 describes a filtration attachment with a large-volume filter insert. The document U.S. 2006/0182657 A1 discloses a combination of a punched filter on which a liquid sample was applied, and a pipette attachment.

The aim of the present invention was therefore to avoid the disadvantages of the prior art by improvements.

According to the invention known porous solid phases with affinity groups are placed from outside in front of the outlet opening of a pipette at right angles to the flow direction of the liquid or they are incorporated into the outlet of an exchangeable tip in order to adsorb nucleic acids or other analytes. As they flow through the solid phase, the nucleic acids or also other analytes are adsorbed under known buffer conditions. The lysate is discharged again and discarded. The analyte adsorbed to the solid phase is further purified by aspirating washing solutions. Finally the analyte is desorbed from the solid phase and collected in a concentrated form in a small elution volume.

The special embodiment of the pipette tip leads to a particularly effective concentration of the analyte. The integration of liquid transfer on the one hand, and adsorption, purification and elution of the analyte on the other hand, enables an improved rapid and simple method. This can not only be used in a manual fashion but can also be implemented in a simple form on known laboratory robots. Current pipette tips for single-channel or multi-channel pipettes or pipette tips which are used in current pipetting robots can be used according to the invention to produce devices according to the invention.

### SUMMARY OF THE INVENTION

In a first aspect the invention comprises a device comprising a pipette (C) and a body with the directly adjoining sections (A) and (B) wherein the body is open at the two sides that are Iodated furthest and opposite to one another and at right angles to the longitudinal axis (L) and allows liquid to flow through along the longitudinal axis through the first opening to the second opening and through this opening;

section (A) comprises an essentially cylindrical wall (100) which is open to one side without constriction and forms the first opening in section (A); the first opening in section (A) is fluidically connected with the outlet opening of the pipette (C); in section (B) the side situated opposite to the first opening is bordered by a further wall (210) wherein the wall (210) adjoins the wall (100) and forms in the middle a circular second opening which is surrounded by an essentially cylindrical wall (200) wherein the wall (200) has a smaller diameter than the wall (100); a disc-shaped filter (600) is arranged at right angles to the longitudinal axis (L) in section (A) and the filter contains a solid phase with affinity groups on its surface (affinity solid phase); in section (B) the side opposite to the first opening is bordered by a further wall (210) where the wall (210) adjoins the wall (100) and in the middle forms a circular second opening which is surrounded by an essentially cylindrical wall (200), where the wall (200) has a smaller diameter than the wall (100); the volume (300) present in section (B) between the filter disc (600) and the second opening is in the range of 0.01% to 5% of the maximum transfer volume of the pipette tip; and in section (B) the diameter of the second opening is in the range of 0.5 mm to 2 mm, characterized in that the first opening in section (A) of the body is fluidically connected to the outlet opening of the pipette (C).

A further aspect of the invention is the use of the device according to the invention to purify nucleic acids.

A further aspect of the invention is a method for purifying nucleic acids using a device according to the invention comprising the steps (a) aspirating and discharging an aqueous lysis buffer containing (i) dissolved nucleic acids and (ii) one or more substances which assist an adsorption of nucleic acids from the liquid phase to a solid phase suitable for the reversible binding of the nucleic acid, wherein the lysis buffer enters the pipette during aspiration through the body with the sections (A) and (B), leaves the pipette again through the body when it is ejected and nucleic acids are adsorbed to the surface of the affinity solid phase during passage of the liquid phase through the filter; (b) aspirating and discharging a washing buffer, wherein the washing buffer contains one or more substances which counteract the detachment of the adsorbed nucleic acids from the solid phase and wherein the washing buffer enters the pipette through the body with the sections (A) and (B) during aspiration, leaves the pipette again through the body when ejected, and impurities pass over into the liquid phase during passage of the liquid phase through the filter; (c) aspirating and discharging an elution buffer, wherein the elution buffer detaches adsorbed nucleic acids from the solid phase and wherein the elution buffer enters the pipette through the body with the sections (A) and (B) during aspiration, leaves the pipette again through the body when ejected, and nucleic acids pass over into the liquid phase during passage of the liquid phase through the filter; (d) collecting the eluate containing the purified nucleic acids.

### BRIEF DESCRIPTION OF THE FIGURES

FIG. 1: Schematic drawing of a cross-section. The lower section of the body of a pipette tip (C) is shown the wall (700) of which can be conically shaped and ends at the outlet (800). The end of the body of the pipette tip is fluidically connected with the upper part of an attachment consisting of the sections (A) and (B) wherein the attachment is open at the top and bottom and allows liquid to flow through. Section (A) comprises a wall (100) with an essentially cylindrical shape which is open at the top. It is connected with the outlet of the pipette tip in such a manner that liquid can be moved out of and/or into the pipette body through the sections (A) and (B) without liquid leaking from the side. The opening facing downwards in section (A) of the hollow body bordered by the wall (100) is bordered in the lower part of the attachment (B) by a further wall (210). This wall adjoins the wall (100) and tapers in the middle to an outlet that is open at the bottom with an inner space (300). The wall (200) which surrounds the outlet after the taper has an essentially cylindrical shape and has a smaller diameter than the wall (100). A disc-shaped filter (600) is arranged at right angles to the longitudinal axis (L) in section (A) and is held above and below by support grids (500), (400) connected to the wall (100). The lower support grid (400) is optional and may be absent. The support grid (400), if present, can also be connected to the wall (210). The filter disc (600) is round and the outer edge of the filter is typically flush with the inner-facing side of the wall (100).

### DETAILED DESCRIPTION OF THE INVENTION

For many bioanalytical processes it is necessary to purify and isolate the analyte to be measured from the sample before the measurement. Especially in the field of nucleic acid isolation, substances from the sample often interfere with the measurement and make the analysis impossible without prior purification of the analyte. Hence, in recent years many methods and commercial kits have been developed for the purification of nucleic acids. These are offered for the manual and automated handling of single samples up to the processing of 96 or even 384 sample batches. The time required for this is from about 10 minutes up to 2.5 hours and more.

Nucleic acids are analytes of considerable importance particularly in medical or forensic diagnostics. Nucleic acids are components of biological material, for example prokaryotic and eukaryotic cells or viruses. In connection with the description of the present invention the term biological material is used synonymously with the term sample material. Apart from the previously mentioned materials, this also encompasses biological liquids such as for example whole blood, serum, plasma, urine, sputum, bone marrow, liquor or lavage. The term sample material also includes biopsy material, fixed and non-fixed tissue samples as well as homogenates thereof and clarified supernatants of such homogenates.

Almost every method of sample preparation includes the transfer of liquids by a pipette tip. It is therefore proposed that a solid phase suitable for immobilizing the analyte is functionally integrated into a pipette tip suitable for transferring liquid volumes. Integration of isolation and purification steps in the necessary transfer of liquid sample material and reagents allows additional vessels e.g. centrifuge columns which contain the solid phase to be omitted. In another embodiment of the prior art using particles as a solid phase it is possible to also omit these particles as well as additional vessels for mixing and separating the particles from the liquid phase.

In a first aspect the invention comprises a device comprising a pipette (C) and a body with the sections (A) and (B). The pipette has an outlet opening and an opening in its upper part wherein liquids are aspirated through the outlet opening into the hollow space of the pipette and can be ejected again from there. The pressure changes required for this act through the opening in the upper part of the pipette.

In order to overcome the described disadvantages of the known functionalized pipette tips a porous thin membrane which has an adequately high binding capacity for the analyte to achieve the required sensitivity is inserted into the cross-section of the pipette tip in such a manner that the analysis solution flows through the entire membrane. The membrane is so thin that no appreciable liquid volume can remain in the membrane pores. Thus, no interfering substances of importance are transferred from the reagents or the sample into the eluate during the following washing steps.

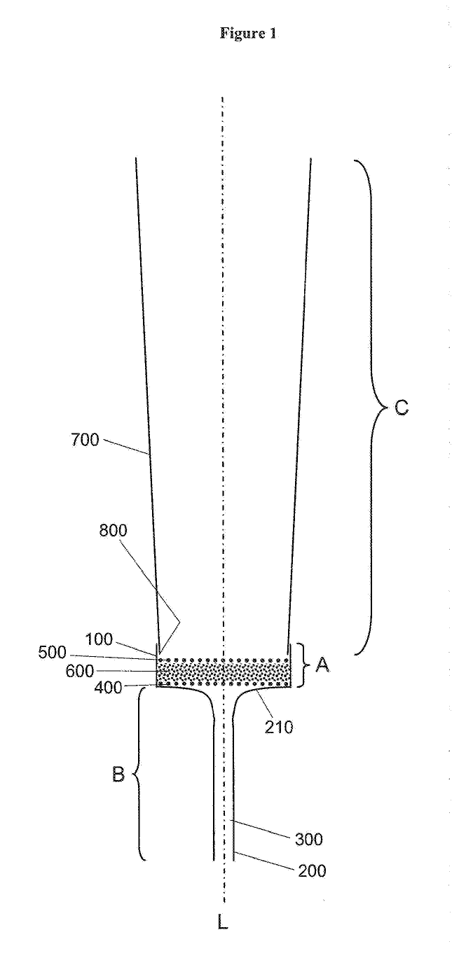

The body in the device according to the invention is divided into the directly adjoining sections (A) and (B). The body is open at the two opposing sides situated furthest from one another and at right angles to the longitudinal axis (L) and allows liquid to flow through along the longitudinal axis through the first opening to the second opening and through this opening. Section (A) comprises an essentially cylindrical wall (100) with a first opening which is open to one side without constriction. The side in section (B) opposite to the first opening is bordered by a further wall (210) wherein this wall (210) adjoins the wall (100) and in the middle forms a circular second opening. The second opening is arranged opposite to the first opening. It is surrounded by an essentially cylindrical wall (200) wherein the wall (200) has a smaller diameter than the wall (100). A disc-shaped filter (600) is arranged at right angles to the longitudinal axis (L) in section (A). The filter contains a solid phase with affinity groups on the surface (affinity solid phase). According to the invention the first opening of the body in section (A) is fluidically connected with the outlet opening (800) of the pipette (C). FIG. 1 illustrates the arrangement of the elements in the device according to the invention according to a preferred embodiment.

It is important for the invention that the nucleic acids should be collected in the smallest possible eluate volume in order to concentrate the analyte to increase the sensitivity and to enable the person responsible for processing to use the eluate as completely as possible in a test. For this purpose the volume of section (B) of the device according to the invention is designed to be as small as possible. Furthermore, one advantageously uses a filter disc (600) which is thin; a disc-shaped filter with a thickness in the range of 0.2 mm to 2 mm is preferred. At the same time the available area of the filter disc should, however, not be too small which for example would be the case were it to be inserted into the conically tapering lower outlet of pipette tips. If the membrane were to be inserted further above in the tip where the full cross-section of the pipette tip is available, the available volume of the pipette tip would be reduced. Also in this case it is not possible to elute in a smallest possible volume because the meniscus of a small liquid volume tears and the negative pressure above the liquid collapses. The liquid would not be transported further through the membrane against gravity.

It is particularly advantageous that, in comparison to the known attachments for pipette tips, a greatly reduced liquid volume can be aspirated to behind the filter and ejected again in the device according to the invention without interruption by entrapped air. In this process the water column aspirated into the device can be sucked in free from bubbles.

Therefore a plastic part is proposed as an embodiment for the functional pipette tip which ends very narrowly in the lower outlet and extends to the required cross-section shortly below the porous membrane. In this connection the lower outlet is so long that it extends to the bottom of conventional deep well microwell plates. Thus, liquid is not displaced in the well of the microwell plate and also the tip does not crash onto the upper rim of the well as the cross-section of the tip increases. In this embodiment it is now possible to take up a small volume of elution solution into the narrow tube and guide it to the membrane through which it is to flow. After the passage the elution solution is ejected into a clean vessel. For production-related reasons it may be necessary for this to manufacture a two-part tip in order to thus introduce the porous membrane with additional supporting grids (see drawing). Alternatively the membrane with a supporting construction can be inserted from the, upper wide opening. In the case of the two-part tip the two parts are clamped and not glued in order to avoid occupancy of the membrane with evaporated materials from adhesives. These evaporated materials prevent the adsorption of nucleic acids onto functional membranes and surfaces.

In section (B) of the body the second opening tapers and, in a preferred embodiment, converges to a narrow channel where the channel ends at the second opening. The volume (300) in section (B) of the body between the filter disc (600) and the second opening is particularly preferably in the range of 0.01% to 10% of the maximum transfer volume of the pipette tip. This volume includes the channel. Even more preferably the volume (300) is in the range of 0.01% to 1% of the maximum transfer volume of the pipette tip. An important advantage of a smallest possible volume (300) is to minimize reagent carry-over. Hence, a narrow short channel is particularly preferred. However, there are limits to the narrowness of the channel with regard to the viscosity of the liquids to be pipetted. The diameter of the channel and of the second opening is particularly preferably in the range of 2 mm to 0.5 mm. These dimensions have proven to be particularly advantageous especially with regard to the viscosity of the solutions that are to be moved by the device according to the invention. Thus, for example solutions with a high content of guanidinium salts that are often used to purify nucleic acids are characterized by an elevated viscosity. A person skilled in the art would in such a case select the diameter of the channel by systematic experimentation such that the passage of the viscous solutions through the device according to the invention is ensured. However, the special effect of the invention becomes evident because the volume (300) remains in the stated preferred ranges i.e. the channel is designed to be shorter when the diameter is wider.

Another important effect of the small volume (300) is that the risk of carry-over is reduced.

The pipette can be connected to the first opening in section (A) of the body in various ways. The connection is configured as a fluidic connection which is why a liquid can be transferred through the first opening in section (A) of the body and the outlet opening of the pipette into the pipette volume. The fluidic connection between the pipette (C) and section (A) is sealing and thus ensures that no liquid escapes at the edges of these elements. In a preferred embodiment the connection of the first opening in section (A) of the body with the outlet opening of the pipette (C) is selected from the group comprising a plug connection, a screw connection, a connection by gluing and a connection by welding.

Especially in the case of plug connections the user will take care that the pressure with which the liquid is moved, is limited in order to prevent the plugged on body from coming off the pipette. Factors which influence the required pipetting pressure are the viscosity of the liquids to be moved as well as the diameter of the channel in section (B) and the width of the second opening.

In a simply designed case the filter (600) can be secured by means of the fact that the edge of the outlet opening of the pipette (C) that is inserted into the first opening presses the filter against the wall (210). In this embodiment no further holder at all is required.

In a further preferred embodiment the filter (600) is held on both sides of the disc by one or two support grids (500), (400) wherein the support grid (400) between the filter (600) and wall (210) is optional. In the case of only one grid, it is attached as a support grid (500) between the filter disc and the first opening. In this case the wall (210) supports the filter (600) from the opposite side. If this arrangement is not sufficient to prevent an undesired displacement of the filter when a liquid phase passes through, a further grid can be attached as a support grid (400) between the filter disc and the wall (210) in a further preferred embodiment. Furthermore, it is preferred that the support grid (500) is connected to the wall (100) and that the support grid (400), if present, is connected to the wall (100) or to the wall (210).

In a further preferred embodiment of the invention the filter (600) is a round (circular) shaped disc. The shape or diameter of this disc is selected such that the outer edge of the filter is essentially flush with the inner-facing side of the wall (100).

The pipette (C) is a tubular body which is entirely conically shaped or conically shaped at the outlet opening and is thus constricted. For the invention it is immaterial whether the inventive device is realized on a single-channel or multi-channel pipette. However, multi-channel pipettes are preferred because they allow a parallel processing of a plurality of samples.

In a preferred embodiment the pipette (C) comprises an exchangeable pipette tip. In this case the outlet opening of the pipette tip is fluidically connected to the first opening in section (A) of the body.

The nucleic acids contained in biological material are usually bound in complexes with proteins and located in compartments from which they have to be released. This takes place by a process which is referred to as lysis. Known lysing methods use aqueous lysis buffers which contain individually or combined one or more chaotropic compounds (the use of guanidinium salts is particularly widespread), detergents, enzymes that degrade cell walls and proteases. Nucleic acids present in the material are exposed and freed of adhering proteins by incubating the biological material in lysis buffer. If the lysate contains insoluble components, they are removed and thus a clarified supernatant is provided.

After the lysis step the dissolved nucleic acids (analyte) can be separated from the lysate by reversible immobilization (adsorption) on the solid phase with a surface that has an affinity for nucleic acids such as silica. When using solid phases with a silica surface it is possible without further ado to use the reagent solutions and process steps that are described in the already known HIGH PURE kits (Roche Diagnostics Operations, Inc.) in an adapted manner.

In a preferred embodiment of the invention the affinity solid phase in the filter in section (A) consists of a fleece with a mineral surface where the surface is suitable for reversibly binding nucleic acids. Preferred surfaces of the affinity solid phase contain silica groups. Porous membranes and reagents for binding analytes are sufficiently well-known to a person skilled in the art. Thus, for example a glass fibre fleece VLS403 known from the HIGH PURE product line (Roche Applied Science, Roche Diagnostics GmbH, Mannheim, Germany) can be used to bind nucleic acids. Further suitable solid phases for binding nucleic acids are for example cellulose membranes or derivatized cellulose membranes whose application is described in U.S. 2005/0112658. Cellulose membranes and derivatives thereof which fulfil the requirements of the invention are also sold by the Schleicher & Schuell Company and are named 0E67 or ST69 and RC60. Furthermore, it is possible to use membranes which are chemically derivatized in such a manner that they carry positively charged groups bound via a linker molecule. These systems are for example described in WO 2004/055213 or EP 1 036 082 (DNA Research Innovation) and U.S. 2005/0106576 (Lumigen). The nucleic acid binds to these positive charges at a suitable pH and under suitable salt conditions as is adequately well-known. After a washing step with buffers that are also known, the nucleic acids are eluted. This can, on the one hand, be carried out by changing the positive charge by the effect of the pH of the elution buffer or by cleaving the linker molecule in a suitable manner.

After separating the solid phase from the lysate, the bound DNA and/or RNA is washed with suitable washing buffers and thus impurities are removed from the bound nucleic acids. Finally the nucleic acids are detached (eluted) from the solid phase with low salt buffer or with water. The composition of the elution buffer usually allows the nucleic acids dissolved therein to be directly used in subsequent bioanalytical measuring methods such as the amplification of a nucleic acid target sequence by means of the polymerase chain reaction (PCR). A small volume of the elution buffer is preferably selected in order to concentrate the nucleic acids. Typically the elution volume is 0.01-10% of the volume of the sample material before adding the lysis reagent.

A further aspect of the invention is thus a method for purifying nucleic acids using a device according to the invention. The method firstly comprises the aspiration and ejection of an aqueous lysis buffer containing nucleic acids and one or more substances dissolved therein which assist the adsorption of the nucleic acids from the liquid phase to the solid phase. This can for example take place in the presence of a guanidinium salt in a concentration range of 0.5 M to 5 M in the lysis buffer. In addition the adsorption of nucleic acids is assisted by the presence of alcohol (preferably ethanol and/or isopropanol) in a preferred concentration range of 1% to 60% [v/v] in the lysis buffer.

During aspiration the lysis buffer enters the pipette through the body with the sections (A) and (B) and leaves it again through the body when it is ejected. In this process the nucleic acids are brought into contact with the surface of the affinity solid phase and are adsorbed thereto during passage of the liquid phase through the filter.

In a subsequent step a washing buffer is aspirated and ejected wherein the washing buffer contains one or more substances which counteract the detachment of the adsorbed nucleic acids from the solid phase, for example ethanol in an aqueous buffer. During aspiration the washing buffer enters the pipette through the body with the sections (A) and (B) and leaves it again through the body when it is ejected. During the washing step impurities pass over into the liquid phase during passage of the liquid phase through the filter.

In a next step an elution buffer is aspirated and ejected wherein the elution buffer detaches adsorbed nucleic acids from the solid phase. During aspiration the elution buffer enters the pipette through the body with the sections (A) and (B) and leaves it again through the body when it is ejected. The nucleic acids are transferred (eluted) into the liquid phase during passage of the liquid phase through the filter. The eluate containing the purified nucleic acids is collected and can be processed further or stored under suitable conditions.

New methods for running test procedures in parallel e.g. the possibility to carry out PCR in microwell plates with 384 or even 1536 wells also require appropriate developments for adapting the sample throughput in the purification of the analyte. The requirements for miniaturization and simplification of working steps is taken especially into account with the aid of the device according to the invention. The proposed format for isolating the analyte allows a rapid and simple handling for individual samples by using a conventional laboratory pipette as well as parallelization by using a multi-channel pipette. The simple automation can be directly transferred using any conventional laboratory robot which contains a pipetting head for exchangeable tips. Thus, a very rapid isolation of the analyte can be carried out when using 96, 384 or 1536 pipetting heads.

In summary the device according to the invention allows a rapid and simple isolation of an analyte from a biological sample because all steps are reduced to the transfer, mixing and incubation of liquids. All separation steps are integrated into the liquid transfer. Parallelization allows a simple upscaling of the sample throughput using available laboratory automation. The laboratory automation manages without additional separation modules such as centrifugation, magnetic separation and vacuum suction which substantially cheapens the instrumentation and makes it less prone to servicing. At the same time the time required is shortened by integrating the immobilization of the analyte in the liquid transfer.

The previous description, the cited publications and the figures elucidate the invention the protective scope of which is derived from the patent claims. The described methods are to be understood as examples which still describe the subject matter of the invention even after modifications.

What is claimed is:

1. A device comprising a pipette and a body, the pipette comprising an outlet opening and the body having directly adjoining sections A and B, wherein:
the body is open at two sides that are located furthest and opposite each other and at right angles to the longitudinal axis and allows liquid to flow along the longitudinal axis from and through a first opening to and through a second opening;
body section A comprises a first essentially cylindrical wall which is open on one side without constriction and forms the first opening;
the first opening in body section A is fluidically connected with the outlet opening of the pipette;
a disc-shaped filter is arranged at right angles to the longitudinal axis in section A and comprises a solid phase with affinity groups on its surface (affinity solid phase);
body section B comprises the second opening, and a portion of section B situated opposite to the first opening is bordered by a further wall adjoining a second essentially cylindrical wall and forms in the middle the circular second opening which is surrounded by the second essentially cylindrical wall wherein the second essentially cylindrical wall has a smaller diameter than the first essentially cylindrical wall;
the volume in section B between the disc-shaped filter and the second opening is in the range of 0.01% to 5% of the maximum transfer volume of the pipette tip; and
in section B the diameter of the second opening is in the range of 0.5 mm to 2 mm.

2. The device according to claim 1, wherein the affinity solid phase in the filter in section A consists of a fleece with a mineral surface where the surface is suitable for reversibly binding nucleic acids.

3. The device according to claim 1, wherein the disc-shaped filter is held by a first support grid between the filter and the first essentially cylindrical wall and an optional second support grid between the filter and the first essentially cylindrical wall or the further wall adjoining the essentially cylindrical wall.

4. The device according to claim 3, wherein the first support grid is connected to the first essentially cylindrical wall and the optional second support grid, if present, is connected to the first essentially cylindrical wall or to the further wall adjoining the essentially cylindrical wall.

5. The device according to claim 1, wherein the filter disc is round and an outer edge of the filter is essentially flush with an inner facing side of the first essentially cylindrical wall.

6. The device according to claim 1, wherein the pipette comprises an exchangeable pipette tip and the outlet opening of the exchangeable pipette tip is fluidically connected with the first opening in section A of the body.

7. The device according to claim 1, wherein the distance between the second opening and the side of the filter disc facing the second opening is in the range of 1.5 cm to 0.1 cm.

8. The device according to claim 1, wherein the volume of section B is in a range of 0.1% to 5% of the maximum transfer volume of the pipette tip.

9. The device according to claim 8, wherein the volume of section B is in the range of 0.5% to 2% of the maximum transfer volume of the pipette tip.

10. The device according to claim 1, wherein the connection of the first opening in body section A with the outlet opening of the pipette is selected from the group consisting of a plug connection, a screw connection, a connection by gluing, and a connection by welding.

11. The device according to claim 6 additionally comprising a multi-channel pipetting head for exchangeable tips.

12. The device according to claim 11, additionally comprising a microwell plate with a plurality of wells.

13. The device according to claim 1, additionally comprising an aqueous lysis buffer containing dissolved nucleic acids and substances which assist an adsorption of nucleic acids from a liquid phase to the surface of the filter.

14. A method for preparing purified nucleic acids using a device according to claim 1 comprising the steps of
(a) aspirating and discharging an aqueous lysis buffer containing dissolved nucleic acids and one or more substances which assist an adsorption of nucleic acids from a liquid phase to a solid phase suitable for reversible binding of the nucleic acids,
wherein the lysis buffer enters the pipette during aspiration through the body with the sections A and B, leaves the pipette again through the body when it is ejected, and nucleic acids are adsorbed onto the surface of the affinity solid phase during passage of the liquid phase through the filter;
(b) aspirating and discharging a washing buffer, wherein the washing buffer contains one or more substances which counteract detachment of the adsorbed nucleic acids from the solid phase, and wherein the washing buffer enters the pipette though the body with the sections A and B during aspiration, leaves the pipette again through the body when ejected, and impurities pass over into the liquid phase during passage of the liquid phase through the filter;
(c) aspirating and discharging an elution buffer, wherein the elution buffer detaches adsorbed nucleic acids from the solid phase,
and wherein the elution buffer enters the pipette through the body with the sections A and B during aspiration, leaves the pipette again through the body when ejected, and nucleic acids pass over into the liquid phase during passage of the liquid phase through the filter; and
(d) collecting an eluate containing the purified nucleic acids.
